(19) United States
(12) Patent Application Publication
(10) Pub. No.: US 2023/0293598 A1
(43) Pub. Date: Sep. 21, 2023

(54) PREVENTION OF RADIATION EXPOSURE ASSOCIATED PATHOLOGY BY TREATMENT WITH UMBILICAL CORD DERIVED REGENERATIVE CELLS

(71) Applicant: Narkeshyo LLC, Miami, FL (US)

(72) Inventor: Amit PATEL, Salt Lake City, UT (US)

(73) Assignee: Narkeshyo LLC, Miami, FL (US)

(21) Appl. No.: 18/184,403

(22) Filed: Mar. 15, 2023

Related U.S. Application Data
(60) Provisional application No. 63/319,878, filed on Mar. 15, 2022.

(51) Int. Cl. A61K 35/51; A61P 39/00; A61K 38/18

(52) U.S. Cl. CPC A61K 35/51; A61P 39/00; A61K 38/1866

(57) ABSTRACT

Disclosed are therapeutic compositions and protocols useful for prevention and/or treatment of radiation induced pathology. In one embodiment, the invention provides the use of regenerative cells derived from subepithelial portion of the umbilical cord for suppression of radiation induced injury including hematopoietic, gastrointestinal, vascular and neurological toxicity. In one embodiment the invention provides the use of derivatives of regenerative cells derived from the subepithelial portion of the umbilical cord for treatment of radiation associated pathology. Said derivatives include conditioned media, conditioned media generated from activated regenerative cells, exosomes, and apoptotic bodies. Treatment and/or prophylaxis of acute and/or chronic radiation syndrome is described in the invention.

### CROSS-REFERENCE TO RELATED APPLICATIONS

This application claims priority to U.S. Provisional Application No. 63/319,878, titled “Prevention of Radiation Exposure Associated Pathology by Treatment with Umbilical Cord Derived Regenerative Cells”, and filed Mar. 15, 2022, which is incorporated by reference in its entirety.

### FIELD OF THE INVENTION

The teachings herein relate to compositions and method of treating radiation exposure in a patient comprising administering a therapeutic dose of regenerative cells.

### BACKGROUND OF THE INVENTION

It is known in the art that exposure to ionizing radiation, depending on dosage, results in damage to rapidly proliferating tissues such as the bone marrow , the gastrointestinal tract, and skin as well as to tissues not known to proliferate such as the central nervous system, lung, heart, liver, kidney and gonads). The type and extent of damage is related to number of exposures to high-energy particles, the total radiation dose received and the quality of the radiation (i.e., photons, electrons and muons, neutrons, a particles, fission fragments, heavy nuclei, etc.).

Acute radiation syndrome (ARS) usually progresses with four clinical stages: prodromal phase, latent phase, manifest phase, and recovery or death. Depending on the amount of radiation absorbed, the symptoms may appear within hours to weeks. The prodromal phase usually starts within 48 hours after radiation exposure, but may last up to 6 days after the exposure and symptoms may include nausea, vomiting, fatigue, autonomic nerve anxiety and loss of consciousness. The latent phase may last from several days to several weeks depending on the amount of radiation exposure, and clinical symptoms may not appear partially or completely. However, at this stage, symptoms such as lymphocytopenia, granulocytopenia, and myelogenous deficiency may occur. Symptoms from the manifest phase may occur with several weeks delay. Symptoms from the manifest phase may include hematopoietic syndrome, gastrointestinal syndrome, cardiovascular syndrome, and neurovascular syndrome depending on the amount of radiation exposure. Patients exposed to extreme amounts of radiation can experience all four of these steps within a few hours and die within a short period of time. Among the acute radiation syndrome, the hematopoietic syndrome may be affected, induced or caused by radiation dose of about 0.7-10 Gy, gastrointestinal syndrome may be affected, induced or caused by radiation dose of about 10-30 Gy, and cardiovascular/neurovascular syndrome may be affected, induced or caused by radiation dose of about 50 Gy. In exemplary embodiments of the present invention, acute radiation syndrome includes syndromes affected, induced or caused, by radiation dose of about 0.1 to 100 Gy, about 0.1 to 80 Gy, about 0.1 to 70 Gy, about 0.1 to 60 Gy, about 0.1 to 50 Gy or about 0.7 or 50 Gy, and may be affected, induced or caused by radiation dose of about 0.1 Gy, about 0.5 Gy, about 0.7 Gy, about 1.0 Gy, about 2.0 Gy.

Radiation-induced injury is not only a major side-effect that complicates radiotherapy in approximately 50% of patients with an abdominal or pelvic malignancy, but is also a major threat during accidental exposure or a targeted terror attack. ARS developing from whole-body or significant partial-body irradiation is associated with induction of hematopoietic (HP), gastrointestinal (GI) and cerebrovascular syndrome as well as cutaneous, pulmonary and cardiac toxicity. Damage to the HP component is known to play a major role in mortality, especially in weakening the immune system so that it cannot fend off infections. Another major source of damage stems primarily from GI damage. Collateral damage to GI epithelium can lead to acute radiation enteritis, which is associated with malabsorption, bleeding, pain, diarrhea and malnutrition. These toxicities prevent optimal cancer treatment and can also lead to chronic complications in patients. The high prevalence of hematopoietic loss and acute radiation enteritis, coupled with the paucity of adequate preventative or therapeutic strategies, underscores the importance of further investigation in this field.

### SUMMARY

The invention is directed to the following numbered aspects:
1. A method of treating radiation exposure comprising administering a therapeutic dose of regenerative cells derived from the subepithelial area of the umbilical cord.
2. The method of aspect 1, wherein said radiation exposure is associated with damage to the hematopoietic system.
3. The method of aspect 2, wherein said damage to the hematopoietic system is reduction in blood cell production.
4. The method of aspect 2, wherein said damage to the hematopoietic system is reduction in hematopoietic stem cells.
5. The method of aspect 2, wherein said damage to the hematopoietic system is reduction suppression of ability of bone marrow stromal cells to produce growth factors.
6. The method of aspect 5, wherein said growth factors whose production is reduced by radiation damage are selected from a group comprising of: BLC, Eotaxin-1, Eotaxin-2, G-CSF, GM-CSF, 1-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TOP, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. ANG, EGF, ENA-78, FGF2, Follistatin, G-CSF, GRO, HGF, IL-6, IL-8, Leptin, MCP-1, MCP-3, PDGFB, PLGF, Rantes, TGFBI, Thrombopoietin, TIMPI, TIMP2, uPAR, VEGF, VEGFD, angiopoietin-1, and angiopoietin-2.
7. The method of aspect 2, wherein said damage to the hematopoietic system is reduction suppression of ability of bone marrow stromal cells to adhere to hematopoietic stem cells.
8. The method of aspect 7, wherein said hematopoietic stem cell expresses the marker CD34.
9. The method of aspect 7, wherein said hematopoietic stem cell expresses the marker CD34 and lacks expression of CD38.
10. The method of aspect 7, wherein said hematopoietic stem cell expresses the marker CD34 and CD133.
11. The method of aspect 7, wherein said hematopoietic stem cell expresses the marker CD34 and CD133 and lacks expression of CD38.
12. The method of aspect 7, wherein said hematopoietic stem cell expresses the marker c-kit.
13. The method of aspect 7, wherein said hematopoietic stem cell expresses the interleukin-3 receptor.
14. The method of aspect 7, wherein said hematopoietic stem cell expresses the interleukin-1 receptor.
15. The method of aspect 7, wherein said hematopoietic stem cell expresses the thrombopoietin receptor.
16. The method of aspect 2, wherein said damage to the hematopoietic system is reduced activity of hematopoietic cells in comparison to age matched controls.
17. The method of aspect 16, wherein said activity is oxygen carrying capacity for erythrocytes.
18. The method of aspect 16, wherein said activity is phagocytosis activity for neutrophils.
19. The method of aspect 16, wherein said activity is generation of hypochlorous acid granules for neutrophils.
20. The method of aspect 16, wherein said activity is phagocytic activity for macrophages.
21. The method of aspect 16, wherein said activity is cytotoxic activity of natural killer cells.
22. The method of aspect 16, wherein said activity is cytotoxic activity for CD8 T cells.
23. The method of aspect 16, wherein said activity is interferon gamma secretion for Th1 cells.
24. The method of aspect 16, wherein said activity is interleukin-4 secretion for Th2 cells.
25. The method of aspect 16, wherein said activity is interleukin-9 secretion for Th9 cells.
26. The method of aspect 16, wherein said activity is interleukin-17 secretion for Th17 cells.
27. The method of aspect 16, wherein said activity is antibody dependent cellular cytotoxicity for monocytes.
28. The method of aspect 16, wherein said activity is antibody dependent cellular cytotoxicity for NK cells.
29. The method of aspect 16, wherein said activity is antibody production for B cells.
30. The method of aspect 16, wherein said activity is ability to form clots for platelets.
31. The method of aspect 16, wherein said activity is ability to inhibit proliferation of activated T cells by T regulatory cells.
32. The method of aspect 1, wherein said radiation exposure is associated with damage to the nervous system.
33. The method of aspect 32, wherein said damage to the nervous system results in one or more behavioral abnormalities.
34. The method of aspect 32, wherein said damage to the nervous system results in nerve conduction defects.
35. The method of aspect 32, wherein said damage to the nervous system results in memory loss.
36. The method of aspect 32, wherein said damage to the nervous system results in suppression of neurogenesis.
37. The method of aspect 1, wherein said radiation exposure is associated with damage to the gastrointestinal system.
38. The method of aspect 1, wherein said radiation exposure is associated with damage to the respiratory system.
39. The method of aspect 1, wherein said radiation exposure is associated with damage to the pulmonary system.
40. The method of aspect 1, wherein said regenerative cells derived from the subepithelial area of the umbilical cord.
42. The method of aspect 1, wherein said regenerative cells derived from the subepithelial area of the umbilical cord comprises an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.
43. The method of aspect 42, wherein said regenerative cells derived from the subepithelial area of the umbilical cord CD29, CD73, CD90, CD166, SSEA4, CD9, CD44, CD146, and CD105.
44. The method of aspect 42, wherein said regenerative cells derived from the subepithelial area of the umbilical cord does not express CD45, CD34, CD14, CD79, CD106, CD86, CD80, CD19, CD117, Stro-1, and HLA-DR.
45. The method of aspect 42, wherein said regenerative cells derived from the subepithelial area of the umbilical cord expresses SOX-2.
46. The method of aspect 42, wherein said regenerative cells derived from the subepithelial area of the umbilical cord expresses OCT-4.
47. The method of aspect 42, wherein said regenerative cells derived from the subepithelial area of the umbilical cord expresses SOX2 and OCT4.
48. The method of aspect 1, wherein derivatives of said regenerative cell are utilized for radioprotection.
49. The method of aspect 48, wherein said derivates are selected form a group comprising of: a) exosomes; b) apoptotic bodies; c) extracellular vesicles; and d) conditioned media.
50. The method of aspect 1, wherein said cell population expresses complement receptor-2 and CD73.
51. The method of aspect 1, wherein said cell population expresses complement receptor-3 and CD73.
52. The method of aspect 1, wherein said cell population expresses complement receptor-4 and CD73.
53. The method of aspect 1, wherein said cells are treated with an activator of an immune receptor.
54. The method of 53, wherein said immune receptor is TLR. 1
55. The method of aspect 54, wherein said TLR-1 is activated by Pam3CSK4.
56. The method of aspect 53, wherein said immune receptor is TLR-2
57. The method of aspect 56, wherein said TLR-2 is activated by HKLM.
58. The method of aspect 53, wherein said immune receptor is TLR-3.
59. The method of aspect 58, wherein said TLR-3 is activated by Poly:IC.
60. The method of aspect 53, wherein said immune receptor is TLR-4.
61. The method of aspect 60, wherein said TLR-4 is activated by LPS.
62. The method of aspect 60, wherein said TLR-4 is activated by Buprenorphine.
63. The method of aspect 60, wherein said TLR-4 is activated by Carbamazepine.
64. The method of aspect 60, wherein said TLR-4 is activated by Fentanyl.
65. The method of aspect 60, wherein said TLR-4 is activated by Levorphanol.

66. The method of aspect 60, wherein said TLR-4 is activated by Methadone.
67. The method of aspect 60, wherein said TLR-4 is activated by Cocaine.
68. The method of aspect 60, wherein said TLR-4 is activated by Morphine.
69. The method of aspect 60, wherein said TLR-4 is activated by Oxcarbazepine.
70. The method of aspect 60, wherein said TLR-4 is activated by Oxycodone.
71. The method of aspect 60, wherein said TLR-4 is activated by Pethidine.
72. The method of aspect 60, wherein said TLR-4 is activated by Glucuronoxylomannan from Cryptococcus.
73. The method of aspect 60, wherein said TLR-4 is activated by Morphine-3-glucuronide.
74. The method of aspect 60, wherein said TLR-4 is activated by lipoteichoic acid.
75. The method of aspect 60, wherein said TLR-4 is activated by beta.-defensin 2.
76. The method of aspect 60, wherein said TLR-4 is activated by low molecular weight hyaluronic acid.
77. The method of aspect 76, wherein said low molecular weight hyaluronic acid has a molecular weight of <1000 kDa.
78. The method of aspect 77, wherein said low molecular weight hyaluronic acid has a molecular weight of <500 kDa.
79. The method of aspect 77, wherein said low molecular weight hyaluronic acid has a molecular weight of <250 kDa.
80. The method of aspect 77, wherein said low molecular weight hyaluronic acid has a molecular weight of <100 kDa.
81. The method of aspect 60, wherein said TLR-4 is activated by fibronectin EDA.
82. The method of aspect 60, wherein said TLR-4 is activated by snapin.
83. The method of aspect 60, wherein said TLR-4 is activated by tenascin C.
84. The method of aspect 53, wherein said immune receptor is TLR-5.
85. The method of aspect 44, wherein said TLR-5 is activated by flaggelin.
86. The method of aspect 53, wherein said immune receptor is TLR-6.
87. The method of aspect 86, wherein said TLR-6 is activated by FSL-1.
88. The method of aspect 53, wherein said immune receptor is TLR-7.
89. The method of aspect 48, wherein said TLR-7 is activated by imiquimod.
90. The method of aspect 83, wherein said immune receptor is TLR-8.
91. The method of aspect 50, wherein said TLR-8 is activated by ssRNA40/LyoVec.
92. The method of aspect 53, wherein said immune receptor is TLR-9.
93. The method of aspect 92, wherein said TLR-9 is activated by a CpG oligonucleotide.
94. The method of aspect 92, wherein said TLR-9 is activated by ODN2006.
95. The method of aspect 92, wherein said TLR-9 is activated by Agatolimod.
96. The method of aspect 92, wherein said TLR-9 is activated by ODN2007.
97. The method of aspect 92, wherein said TLR-9 is activated by ODN1668.
98. The method of aspect 92, wherein said TLR-9 is activated by ODN1826.
100. The method of aspect 92, wherein said TLR-9 is activated by one or more compounds selected from a group comprising of: ODN BW006, ODN D SL01, ODN 2395, ODN M362 and ODN SL03.
101. The method of aspect 1, wherein said cells are extracted from Wharton Jelly and possess immune modulatory, neurogenic, anti-inflammatory, angiogenic and regenerative activity.
102. The method of aspect 1, wherein said cells are plastic adherent.
103. The method of aspect 1, wherein said cells express interleukin-3 receptor and CD73.
104. The method of aspect 1, wherein said cells express interleukin-3 receptor and CD37.
105. The method of aspect 1, wherein said cells express interleukin-3 receptor and CD69.
106. The method of aspect 1, wherein said cells express interleukin-3 receptor and surface vimentin.
107. The method of aspect 1, wherein said cells express GM-CSF receptor.
108. The method of aspect 1, wherein said cells express GM-CSF receptor and CD73.
109. The method of aspect 1, wherein said cells express GM-CSF receptor and CD37.
110. The method of aspect 1, wherein said cells express GM-CSF receptor and CD69.
111. The method of aspect 1, wherein said cells express GM-CSF receptor and surface vimentin.
112. The method of aspect 1, wherein said cells express surface vimentin and one or more of the following markers: a) CD29; b) CD36; c) CD37; d) CD73; e) CD90; f) CD166; g) SSEA4; h) CD9; i) CD44; k) CD146; 1) CD105; and m) HLA-G
113. The method of aspect 112, wherein said cells possess ability to generate soluble TNF-alpha receptor at a concentration of 10 pg-1 ng per 1,000,000 cells under basal growth conditions in DMEM media with 10% fetal calf serum.
114. The method of aspect 112, wherein said cells possess ability to generate soluble TNF-alpha receptor at a concentration of 10 pg-1 ng per 1,000,000 cells under basal growth conditions in DMEM media with 10% fetal calf serum.
115. The method of aspect 112, wherein said cells possess ability to generate soluble TNF-alpha receptor at a concentration of 50 pg-500 pg per 1,000,000 cells under basal growth conditions in DMEM media with 10% fetal calf serum.
116. The method of aspect 112, wherein said cells possess ability to generate soluble HLA-G receptor at a concentration of 1 pg-10 ng per 1,000,000 cells under basal growth conditions in DMEM media with 10% fetal calf serum.
117. The method of aspect 112, wherein said cells possess ability to generate soluble HLA-G receptor at a concentration of 10 pg-1 ng per 1,000,000 cells under basal growth conditions in DMEM media with 10% fetal calf serum.
118. The method of aspect 112, wherein said cells possess ability to generate soluble HLA-G at a concentration of 50 pg-500 ng per 1,000,000 cells under basal growth conditions in DMEM media with 10% fetal calf serum.
119. The method of aspect 112, wherein said cells possess ability to generate soluble HLA-G at a concentration of 10 pg-1 ng per 1,000,000 cells under basal growth conditions in DMEM media with 10% fetal calf serum.
120. The method of aspect 112, wherein said cells possess ability to generate interleukin 10 at a concentration of 1 pg-10 ng per 1,000,000 cells under basal growth conditions in DMEM media with 10% fetal calf serum.
121. The method of aspect 112, wherein said cells possess ability to generate interleukin 10 at a concentration of 10 pg-1 ng per 1,000,000 cells under basal growth conditions in DMEM media with 10% fetal calf serum.
122. The method of aspect 112, wherein said cells possess ability to generate interleukin 10 at a concentration of 50 pg-500 ng per 1,000,000 cells under basal growth conditions in DMEM media with 10% fetal calf serum.
123. The method of aspect 112, wherein said cells possess ability to generate interleukin 10 at a concentration of 50 pg-200 ng per 1,000,000 cells under basal growth conditions in DMEM media with 10% fetal calf serum.
124. The method of aspect 112, wherein said cells possess ability to generate interleukin 35 at a concentration of 1 pg-10 ng per 1,000,000 cells under basal growth conditions in DMEM media with 10% fetal calf serum.
125. The method of aspect 112, wherein said cells possess ability to generate interleukin 35 at a concentration of 10 pg-1 ng per 1,000,000 cells under basal growth conditions in DMEM media with 10% fetal calf serum.
126. The method of aspect 112, wherein said cells possess ability to generate interleukin 35 at a concentration of 50 pg-500 ng per 1,000,000 cells under basal growth conditions in DMEM media with 10% fetal calf serum.
127. The method of aspect 112, wherein said cells possess ability to generate interleukin 35 at a concentration of 50 pg-200 ng per 1,000,000 cells under basal growth conditions in DMEM media with 10% fetal calf serum.

128. The method of aspect 112, wherein said cells possess ability to generate interleukin 4 at a concentration of 1 pg-10 ng per 1,000,000 cells under basal growth conditions in DMEM media with 10% fetal calf serum.
129. The method of aspect 112, wherein said cells possess ability to generate interleukin 4 at a concentration of 10 pg-1 ng per 1,000,000 cells under basal growth conditions in DMEM media with 10% fetal calf serum.
130. The method of aspect 112, wherein said cells possess ability to generate interleukin 4 at a concentration of 50 pg-500 ng per 1,000,000 cells under basal growth conditions in DMEM media with 10% fetal calf serum.
131. The method of aspect 1, wherein said cells are isolated by selection for markers selected from a group comprising of: CD39, CD73, FOXP3, GITR, CLTA4, ICOS, GARP, LAP, PD-1, CCR6, and CXCR3.
132. The method of aspect 131, wherein said cell population expresses OCT-4.
133. The method of aspect 131, wherein said cell population expresses SOX-2.
134. The method of aspect 131, wherein said cell population expresses NANOG.
135. The method of aspect 131, wherein said cell population expresses c-Met.
136. The method of aspect 131, wherein said cell population expresses PDGF receptor.
137. The method of aspect 131, wherein said cell population expresses OCT-4.
138. The method of aspect 1, wherein said cell population expresses CD13 and CD73.
139. The method of aspect 1, wherein said cell population expresses CD29 and CD73.
140. The method of aspect 1, wherein said cell population expresses CD54 and CD73.
141. The method of aspect 1, wherein said cell population expresses SSEA4 and CD73.
142. The method of aspect 1, wherein said cell population expresses CD31 and CD73.
143. The method of aspect 1, wherein said cell population expresses CD34 and CD73.
144. The method of aspect 1, wherein said cell population expresses TNF-alpha receptor p55 and CD73.
145. The method of aspect 1, wherein said cell population expresses TNF-alpha receptor p75 and CD73.
146. The method of aspect 1, wherein said cell population expresses interleukin-1 beta receptor and CD73.
147. The method of aspect 1, wherein said cell population expresses interleukin-6 receptor and CD73.
148. The method of aspect 1, wherein said cell population expresses interleukin-8 receptor and CD73.
149. The method of aspect 1, wherein said patient exposed to radiation is also treated in a manner to alter the gut microbiome in order to obtain enhanced protective and/or regenerative effects from radiation exposure.
150. The method of aspect 149, wherein said subject is administered a bacterium and/or metabolite thereof, wherein the bacterium comprises one or more bacterial strains capable of producing short chain fatty acids (SCFAs).
151. The method of aspect 150, wherein the bacterium comprises intestinal microbiota.
152. The method of aspect 150, wherein the SCFAs produced by the bacterial strains comprise acetate, butyrate and propionate, optionally wherein the ratio of acetate to butyrate to propionate is about 1:5:50, optionally about 1:5:100.
153. The method of any of aspects 151 to 152, wherein the bacterium comprises strains selected from Lachnospiraceae, Enterococcus faecalis, Lactobacillus rhamonosusl, and combinations thereof.

154. The method of any of aspects 151 to 153, wherein the bacterium comprises Lachnospiraceae strains, optionally wherein the Lachnospiraceae strains produce butyrate higher than about 120 .mu.M and propionate higher than about 60 .mu.M.
155. The method of any of aspects 151 to 154, wherein the metabolite comprises one or more tryptophan metabolites.
156. The method of any of aspects 151 to 155, wherein the subject is suffering from acute radiation syndrome (ARS), hematopoietic (HP) injury, gastrointestinal (GI) injury, cerebrovascular syndrome, cutaneous toxicity, pulmonary toxicity, cardiac toxicity and/or combinations thereof.
157. The method of any of aspects 151 to 156, wherein administration of the bacterium and/or metabolite thereof effectively attenuates radiation-induced hematopoietic and/or gastrointestinal syndrome.
158. The method of any of aspects 151 to 157, wherein the administration of the bacterium and/or metabolite to the subject occurs before or after radiation exposure.
159. The regenerative cell population of aspect 1, produced by the following steps: a) obtaining an isolated umbilical cord; b) dissociating Wharton's Jelly to obtain mononuclear cells; c) optionally purifying subsets of said mononuclear cells; d) exposing said mononuclear cells to a regenerative adjuvant; e) culturing said mononuclear cells in absence of, or in the presence of said regenerative adjuvant and/or adding a secondary regenerative adjuvant; and f) selecting said cultured cells for further use, optionally carrying out another purification step.
160. The composition of aspect 1, wherein said cell isolated from said Wharton's Jelly express a marker selected from a group of markers comprising: CD144, CD105, and CD31.
161. The composition of aspect 1, wherein said cells are obtained from placenta perivascular tissue as a substitute for Wharton's Jelly.
162. The composition of aspect 161, wherein said cells are isolated from fetal vascular lobules of a hemochorial placenta.
163. The composition of aspect 161, wherein said cells are isolated by: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45, optionally one or more steps are performed in the presence of hypoxia, wherein hypoxia is sufficient to induce translocation of HIF-1 alpha.
164. The method of aspect 163, wherein dissociation of fetal vascular lobes is accomplishing by incubation with a mixture of about 2% collagenase, about 0.25% trypsin and about 0.1% DNAse in tissue culture medium.
165. The method of aspect 18=63, wherein dissociation of fetal vascular lobes is accomplishing by incubation with a mixture of about 2% collagenase, about 0.25% trypsin and about 0.1% DNAse in tissue culture medium.
166. The composition of aspect 1, wherein cells isolated are comprised of adherent cells expressing the marker CD73 but substantially lacking CD105.
167. The composition of aspect 1, wherein cells isolated are comprised of adherent cells expressing the marker CD73 and CD105 but lacking in CD90.
168. The composition of aspect 1, wherein said regenerative adjuvant is an anti-inflammatory cytokine.
169. The composition of aspect 168, wherein said anti-inflammatory cytokine is selected from a group comprising of IL-4, IL-10, IL-13, IL-20, IL-22 and IL-35.
170 The composition of aspect 168, wherein said anti-inflammatory cytokine is TGF-beta.
171. The composition of aspect 168, wherein said anti-inflammatory cytokine is PGE-2.
172. The composition of aspect 168, wherein said anti-inflammatory cytokine is VEGF.
173. The composition of aspect 168, wherein said composition is composed of mesenchymal stem cells, and wherein said first regenerative adjuvant is hypoxia.
174. The composition of aspect 1, wherein said mesenchymal stem cells possess one or more markers selected from a group comprising of: a) CD11b; b) CD11c; c) CD20; d) CD56; e) CD57 f) CD73; g) CD90; h) CD105; i) membrane bound TGF-beta; and j) neuropilin.
175. The composition of aspect 1, wherein said composition is capable of inhibiting T cell mediated immune responses.
176. The method of aspect 175, wherein said T cell mediated immune responses comprise of Th1 cell production of cytokines.
177. The method of aspect 176, wherein said cytokine is selected from a group of cytokines comprising of: a) IL-2; b) IL-6; c) IL-8; d) IL-12; e) IL-15; f) IL-18; g) interferon gamma; h) TNF-alpha; and i) interleukin-33.
178. The method of aspect 176, wherein said T cell mediated immune responses is activation of gamma delta T cells.
179. The method of aspect 178, wherein said gamma delta T cell activation is production of granzyme.
180. The method of aspect 178, wherein said gamma delta T cell activation is production of perforin.
181. The method of aspect 178, wherein said gamma delta T cell activation is production of interferon gamma.
182. The method of aspect 178, wherein said T cell mediated immune response is proliferation of a T cell.
183. The method of aspect 178, wherein said T cell mediated immune response is activation of a CD8 T cell.
184. The method of aspect 183, wherein said activation of CD8 cell is proliferation of said CD8 cell.
185. The method of aspect 183, wherein said activation of CD8 cell is production of perforin.
186. The method of aspect 183, wherein said activation of CD8 cell is production of granzyme.
187. The method of aspect 183, wherein said activation of CD8 cells is induction of cytotoxicity.
188. The method of aspect 183, wherein said activation of CD8 cells is production of inflammatory cytokines.
189. The method of aspect 188 wherein said inflammatory cytokines are selected from a group comprising of: a) RANTES; b) MIP-1 alpha; c) MIP-1 beta; d) IL-2; e) IL-6; f) IL-8; g) IL-12; h) IL-15; i) IL-18; j) interferon gamma; k) TNF-alpha; and 1) interleukin-33.
190. The method of aspect 183, wherein said T cell mediated immune response is a homeostatic expansion of said T cells.
191. The method of aspect 190, wherein said homeostatic expansion comprises proliferation of a T cell in absence of need for a second signal.
145. The method of aspect 144, wherein said second signal is CD28.
146. The method of aspect 144, wherein said second signal is ICOS.
147. The method of aspect 144, wherein said second signal is CD40.
148. The method of aspect 143, wherein said homeostatic expansion is ability of said T cell to proliferate independent of T cell receptor ligation.
149. The method of aspect 143, wherein said homeostatic expansion is ability of said T cell to proliferate in response to IL-7.
150. The method of aspect 143, wherein said homeostatic expansion is ability of said T cell to proliferate in response to IL-15.
151. The method of aspect 128, wherein said inhibition of T cell mediated responses is performed by augmentation of immune regulatory cells.
152. The method of aspect 151, wherein said immune regulatory cells are cells possessing ability to suppress T cell activation, and/or proliferation, and/or cytokine secretion.
153. The method of aspect 151, wherein said immune regulatory cells are T cells.
154. The method of aspect 153, wherein said immune regulatory T cells are T regulatory cells.
155. The method of aspect 154, wherein said T regulatory cells possess ability to suppress a conventional T cell in an antigen specific manner.
156. The method of aspect 154, wherein said T regulatory cells possess ability to suppress a conventional T cell in an antigen non-specific manner.
157. The method of aspect 154, wherein said T regulatory cells possess ability to suppress a conventional T cell in a contact dependent manner.
158. The method of aspect 154, wherein said T regulatory cells possess ability to suppress a conventional T cell in a contact independent manner.
159. The method of aspect 154-158, wherein said T regulatory cells are CD4 positive and CD25 positive.
160. The method of aspect 154-158, wherein said conventional T cells are CD4 positive and CD25 negative.
161. The method of aspect 154-158, wherein said T regulatory cells are CD4 positive and CTLA-4 positive.
162. The method of aspect 154-158, wherein said conventional T cells are CD4 positive and CTLA-4 negative.
163. The method of aspect 154-158, wherein said T regulatory cells are CD4 positive and GITR positive.
164. The method of aspect 154-158, wherein said conventional T cells are CD4 positive and GITR negative.
165. The method of aspect 154-158, wherein said T regulatory cells are CD4 positive and CD39 and/or CD73 positive.
166. The method of aspect 154-158, wherein said conventional T cells are CD4 positive and CD39 and/or CD73 negative.
167. The method of aspect 154-158, wherein said T regulatory cells are CD4 positive and IL-7 receptor negative
168. The method of aspect 154-158, wherein said conventional T cells are CD4 positive and IL-7 receptor positive.
169. The method of aspect 164, wherein said T regulatory cells are capable of inhibiting maturation of dendritic cells in response to ligation of a “danger signal”.
170. The method of aspect 169, wherein said “danger signal” is activation of a toll like receptor (TLR).
171. The method of aspect 170, wherein said TLR is TRL-4.
172. The method of aspect 170, wherein said activation of dendritic cell maturation endows said dendritic cell ability to induce proliferation of naïve T cells.
173. The method of aspect 170, wherein said activation of dendritic cell maturation endows said dendritic cell ability to induce cytokine secretion of naïve T cells.
174. The method of aspect 170, wherein said activation of dendritic cell maturation endows said dendritic cell ability to induce cytotoxic activity to naïve T cells.
175. The method of aspect 170, wherein said activation of dendritic cell maturation endows said dendritic cell ability to induce differentiation of naïve CD4 T cells into a helper phenotype.
176. The method of aspect 175, wherein said helper phenotype is T helper 1, characterized by expression of interferon gamma, interleukin 2, interleukin 7, interleukin 12, interleukin 15, interleukin 18, and interleukin 23.
177. The method of aspect 176, wherein said Th1 cells is characterized by expression of STAT4.
178. The method of aspect 175, wherein said helper phenotype is T helper 2, characterized by expression of IL-4, IL-5, IL-13, and IL-10.
179. The method of aspect 178, wherein said T helper 2 cell is characterized by expression of STAT6.
180. The method of aspect 175, wherein said T helper cell is a Th9 cell.
181. The method of aspect 180, wherein said Th9 cell is characterized by expression of the FOXO1 transcription factor.
182. The method of aspect 180, wherein said Th9 cell is characterized by expression of interleukin-9
183. The method of aspect 175, wherein said T helper cell is a Th17 cell.
184. The method of aspect 183, wherein said Th17 cell produces cytokines selected from a group comprising of: a) IL-17; b) IL-17A); c) IL-17F, and d) IL-6.
185. The method of aspect 183, wherein said Th17 cell expresses BATF.
186. The method of aspect 183, wherein said Th17 cell expresses RORgamma.
187. The composition of aspect 101, wherein said composition is capable of inhibiting antigen presenting cell function.
188. The composition of aspect 187, wherein said antigen presenting cell is a B cell.
189. The composition of aspect 188, wherein said B cell is a CD5 positive B cell.
190. The composition of aspect 187, wherein said antigen presenting cell is an endothelial cell.
191. The composition of aspect 187, wherein said endothelial cell is activated with interferon gamma.
192. The composition of aspect 187, wherein said antigen presenting cell is an epithelial cell.
193. The composition of aspect 187, wherein said antigen presenting cell is activated with interferon gamma.
194. The composition of aspect 187, wherein said antigen presenting cell is a monocyte.
195. The composition of aspect 187, wherein said antigen presenting cell is a macrophage.
196. The composition of aspect 195, wherein said antigen macrophage is an M1 macrophage.
197. The composition of aspect 195, wherein said antigen macrophage is an M2 macrophage.
198. The composition of aspect 196, wherein said M1 macrophage expresses markers selected from a group comprising of: CD80, CD86, CD64, CD16 and CD32.
199. The composition of aspect 196, wherein said M2 macrophage expresses markers selected from a group comprising of: CD68. CD163, and CD206.

### BRIEF DESCRIPTION OF THE DRAWINGS

FIG. 1 is a bar graph showing cells derived from the subepithelial area of the umbilical cord protect from radiation induced death.

FIG. 2 is a bar graph showing cells derived from the subepithelial area of the umbilical cord stimulates hematopoietic cytokine VEGF.

FIG. 3 is a bar graph showing cells derived from the subepithelial area of the umbilical cord stimulates HGF-1.

FIG. 4 is a bar graph showing cells derived from the subepithelial area of the umbilical cord stimulates angiopoietin.

FIG. 5 is a bar graph showing cells derived from the subepithelial area of the umbilical cord stimulates M-CSF.

FIG. 6 is a bar graph showing cells derived from the subepithelial area of the umbilical cord stimulates G-CSF.

FIG. 7 is a bar graph showing cells derived from the subepithelial area of the umbilical cord stimulates hematopoietic cytokine GM-CSF.

### DETAILED DESCRIPTION OF THE INVENTION

The invention provides means of treating radiation exposure through administration of therapeutic regenerative cells derived from the subepithelial portion of the umbilical cord. Administration of said regenerative cells can be performed prophylactically before exposure to radiation, and/or subsequently after exposure to radiation.

“Ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

“Mesenchymal stem cell” The reason to use MSC as compared to other cell types for development of therapeutics is attractive our opinion, because of the following: a) MSC are true “repair cells”. In many conditions, healing is associated with MSC producing growth factors, which coordinate numerous cell types to initiate and maintain recovery of bodily function after injury; b) MSC do not require matching between donor and recipient. This is very important because it allows for the use of the stem cell as a “drug”. This means that large quantity of cells can be grown, standardized, characterized and subsequently used to treat a variety of different patients; c) MSC act as “biological anti-inflammatories”. Specifically, not only do MSC promote healing of injured tissues, but they also reduce inflammation. One very important aspect of MSC reducing inflammation is that they only reduce inflammation in the presence of tissue injury. This means that if MSC are injected into a patient with no inflammation, then the MSC do not produce anti-inflammatory products. In other cases, when the patient suffers from an inflammatory condition, the MSC actually produce more anti-inflammatory agents in order to reduce the inflammation. Essentially, the MSC act as a “natural” anti-inflammatory cell, specifically “knowing” how many and what factors to producing; d) MSC home to tissues of injury. It is known that most injury is associated with generation of blood clotting. This process results in lack of oxygen to the damaged tissue. When tissue lacks oxygen, cells of the tissue start producing “chemokines” which act as local beacons, calling in MSC only to the area of tissue injury. In addition to conceptual advantages of MSC, one very important aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, or intramuscular, have largely not been observed.

“Subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

“Preventing” or “protecting” means preventing in whole or in part, or ameliorating or controlling.

“Treating” refers to both therapeutic treatment and prophylactic or preventative measures, or administering an agent suspected of having therapeutic potential.

“Pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

“Effective amount” refers to an amount of an active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

“Disease”, “disorder” or “condition” refer to a state of being or health status of a patient or subject capable of being treated with the compounds or methods provided herein. As used herein, the term “acute radiation syndrome” or “ARS” refers to a disease or disorder associated with radiation toxicity or radiation sickness (e.g., acute radiation syndrome (ARS) including hematopoietic (bone marrow) acute radiation syndrome, gastrointestinal acute radiation syndrome, cutaneous acute radiation syndrome, cardiovascular acute radiation syndrome, and/or central nervous system (CNS) acute radiation syndrome). The term the term “acute radiation syndrome” or “ARS” also may include radiation-induced coagulopathy. In certain embodiments, the disease is acute radiation syndrome (ARS). In certain embodiments, ARS occurs in a subject upon exposure to a radiation of about 0.1 Gy (or 10 rads) or greater, about 0.2 Gy (or 20 rads) or greater, about 0.3 Gy (or 30 rads) or greater, about 0.4 Gy (or 40 rads) or greater, about 0.5 Gy (or 50 rads) or greater, about 0.6 Gy (or 60 rads) or greater, about 0.7 Gy (or 70 rads) or greater, about 0.8 Gy (or 80 rads) or greater, about 0.9 Gy (or 90 rads) or greater, about 1 Gy (or 100 rads) or greater, about 2.0 Gy(or 200 rads), about 3.0 Gy(or 300 rads) or about 4.0 Gy (or 400 rads) or greater.

In certain embodiments, acute radiation syndrome (ARS) may occur in a subject upon exposure to radiation (including gamma radiation) of about 1 Gy (or 100 rads) to about 8 Gy (or 800 rads) for any varying time periods such as at least 1, 2, 5, 10, 30, 60, 80, 120, 180, 240 or 300 seconds. ARS is described in the literature as possessing hematopoietic , vascular, neurological, pulmonary and other toxicities. The invention aims to prevent and/or treat said toxicities through administration umbilical cord subepithelial cells, and/or derivatives of said cells, and/or said co-administration of said cells with therapeutic adjuvants and/or by pre-priming of said cells before administration.

In some embodiments the invention teaches the use of umbilical cord subepithelial regenerative cells together with various therapeutic adjuvants to protect tissue from radiation damage. In one embodiment cells are administered together with melatonin. One study demonstrating efficacy of melatonin utilized C57BL/6 male mice that were exposed to 2, 5, and 7.5 Gy of whole-body irradiation (WBI), 30 min after intra-peritoneal administration of melatonin with different doses. Mice were sacrificed at different time intervals after WBI, and bone marrow, splenocytes, and peripheral blood lymphocytes were isolated for studying various parameters including micronuclei (MN), cell cycle, comet, γ-H2AX, gene expression, amino acid profiling, and hematology. It was found that Melatonin100 mg/kg ameliorated radiation (7.5 Gy and 5 Gy) induced MN frequency and cell death in bone marrow without mortality. At 24 h of post-WBI (2 Gy), the frequency of micronucleated polychromatic erythrocytes (mnPCE) with different melatonin doses revealed 20 mg/kg as optimal i.p. dose for protecting the hematopoietic system against radiation injury. In comet assay, a significant reduction in radiation-induced % DNA tail was observed at this dose. Melatonin reduced γ-H2AX foci/cell and eventually reached to the control level. Melatonin also decreased blood arginine levels in mice after 24 h of WBI. The gene expression of G-CSF, Bcl-2-associated X protein (BAX), and Bc12 indicated the role of melatonin in G-CSF regulation and downstream pro-survival pathways along with anti-apoptotic activity [1]. Others have reported some benefit of melatonin in reducing toxicity of radiation exposure [2-20]. Melatonin has been shown protective against gastrointestinal pathology induced by radiation [21-28], as well as lung damage [29-32], ear damage [33, 34], oral damage [35-37], muscle damage [38], hematopoietic damage [39-41], gonadal damage [42-45], thyroid damage [46], kidney damage [47, 48], neuronal damage [49-53], hepatic damage [54],. Utilization of melatonin has been reported to induce anti-inflammatory [55, 56], antifibrotic, and immune stimulatory activity [57], which according to the current invention will synergize with cells of the invention to enhance radiation protection.

In another embodiment stem cells of the invention are administered together thymoquinone to protect from radiation [58]. Other “radioprotective adjuvants” are disclosed in the invention that may be utilized together with the cells of the invention in order to obtain synergistic activities in protection from radiation induced pathology, these include Gamma-tocotrienol [59-80], pentoxifylline [81, 82], amifostine [83-85], arbutin [86-90], G-CSF, GM-CSF, interleukin-12, and kelulut honey [91].

The present disclosure provides various cells, stem cells, and stem cell components for treatment of ARS, including associated methods of generating and using such cells. In one aspect, for example, an isolated cell that is capable of self-renewal and culture expansion and is obtained from a subepithelial layer of a mammalian umbilical cord tissue is provided. Such an isolated cell expresses at least three cell markers selected from CD29, CD73, CD90, CD166, SSEA4, CD9, CD44, CD146, or CD105, and does not express at least three cell markers selected from CD45, CD34, CD14, CD79, CD106, CD86, CD80, CD19, CD117, Stro-1, or HLA-DR. In another aspect, the isolated cell expresses CD29, CD73, CD90, CD166, SSEA4, CD9, CD44, CD146, and CD105. In yet another aspect, the isolated cell does not express CD45, CD34, CD14, CD79, CD106, CD86, CD80, CD19, CD117, Stro-1, and HLA-DR. In some aspects, the isolated cell can be positive for SOX2, OCT4, or both SOX2 and OCT4. In a further aspect, the isolated cell can produce exosomes expressing CD63, CD9 or both. It is understood that the present scope includes cultures of isolated cells. The cells according to aspects of the present disclosure are capable of differentiation into a variety of cell types, and any such cell type is considered to be within the present scope. Non-limiting examples of such cell types can include adipocytes, chondrocytes, osteocytes, cardiomyocytes, endothelial cells, myocytes, and the like, including combinations thereof. A variety of cells and cellular products can be derived from the isolated cells described herein, and any such cells and cellular products are considered to be within the present scope. In one aspect, for example, the present disclosure provides an isolated exosome derived from the isolated cells described, where the exosome expresses CD63, CD9 or both. In another aspect, an adipocyte cell that has been differentiated from the isolated cells described is provided. In yet another aspect, a chondrocyte cell that has been differentiated from the isolated cells described is provided. In a further aspect, an osteocyte cell that has been differentiated from the isolated cells described is provided. In yet a further aspect, a cardiomyocyte cell that has been differentiated from the isolated cells described is provided. Furthermore, a culture of differentiated cells derived from the isolated cells described including at least one cell type selected from an adipocyte, a chondrocyte, an osteocyte, or a cardiomyocyte is provided. In another aspect, the present disclosure provides a method of culturing stem cells from a subepithelial layer of a mammalian umbilical cord. Such a method can include dissecting the subepithelial layer from the umbilical cord, placing the dissected subepithelial layer interior side down on a substrate such that an interior side of the subepithelial layer is in contact with the substrate, and culturing the subepithelial layer on the substrate. The method can additionally include removing explants for primary cell expansion. In one aspect, dissecting the subepithelial layer further includes removing Wharton's Jelly from the umbilical cord. The subepithelial layer can be cultured in any media capable of producing explants therefrom, and any such medium is considered to be within the present scope. In one specific aspect, however, one such culture medium can include a platelet lysate. In another aspect, the culture media can include human or animal platelet lysate. In yet another aspect, the culture media can be derived from human-free and animal-free ingredients.

The substrate utilized to culture the subepithelial layer can be any substrate capable of deriving explants therefrom. In one aspect, the substrate can be a polymeric matrix. One example of such a polymeric matrix is a culture dish. In one specific aspect, the culture dish can be a cell culture treated plastic, and the subepithelial layer can be placed thereon without any additional pretreatment to the cell culture treated plastic. In another aspect, the substrate can be a semi-solid cell culture substrate. Any type of semi-solid substrate that is capable of supporting the subepithelial layer during the culturing procedure is considered to be within the present scope.

Various culturing conditions are contemplated, and it is understood that such conditions can vary depending on experimental protocol and various desired results. In one aspect, for example, the subepithelial layer can be cultured in a normoxic environment. In another aspect, the subepithelial layer can be cultured in a hypoxic environment. Additionally, in some aspects, the culturing of the subepithelial layer and the removal of the explants can be performed without the use of any enzymes. Furthermore, in some aspects, subculturing of the explants and/or the cells resulting from the explants can be performed without the use of any enzymes.

The cells of the invention are cultured under hypoxia, in one embodiment, cultured in order to induce and/or augment expression of chemokine receptors. One such receptor is CXCR-4. The population of cells, including population of umbilical cord mesenchymal cells, and more specifically, cells extracted from the subepithelial layer (SL) of the umbilical cord. A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate. A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material. Following placement of the SL on the substrate, the SL is cultured in a suitable medium. In some aspects it is preferable to utilized culture media that is free of animal and human components or contaminants. The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1.times. glutamine, 1.times. NEAA, and 0.1-20% PRP lysate or platelet lysate. Another example of suitable media can include a base medium of DMEM low glucose without phenol red, 1.times. glutamine, 1.times. NEAA, 1000 units of heparin and 20% PRP lysate or platelet lysate. In another example, cells can be cultured directly onto a semi-solid substrate of DMEM low glucose without phenol red, 1.times. glutamine, 1.times. NEAA, and 20% PRP lysate or platelet lysate. In a further example, culture media can include a low glucose medium (500-1000 mg/mL) containing 1.times. Glutamine, 1.times. NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 1.times. Glutamine, 1.times. NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

In other embodiments of the invention, purified populations of regenerative cells can be obtained from the lining of a human umbilical cord. As used herein, “purified” means that at least 90% (e.g., 91, 92, 93, 94, 95, 96, 97, 98, or 99%) of the cells within the population are regenerative cells. As used herein, “regenerative cells” refers to mammalian cell. Within the context of the current invention regenerative cells can be isolated from umbilical cords obtained with informed consent. Typically, after an umbilical cord is obtained in a hospital or clinic, the cord is placed in a hypothermic preservation solution, such as FRS solution from Biolife Solutions (catalog #HTS-FRS) and stored at 4.degree. C. To begin isolating ULSCs, the hypothermic preservation solution can be removed by washing in a buffer, such as Hank's basic salt solution, that is free of Mg.sup.2+, Ca.sup.2+, and phenol free. The umbilical cord can be cut into cross sections in the presence of a buffer, and then the cross-sections can be cut longitudinally into two pieces while avoiding any venous or arterial tissue. If any blood is released into the buffer while cutting the cord, the contaminated buffer is replaced with fresh buffer. The longitudinal pieces of cord can be dissected to remove venous and arterial tissue such that the resulting cord lining (i.e., the gelatinous cord material) is substantially free of venous and arterial tissue. As used herein “substantially free of venous and arterial tissue” indicates that as much visible venous and arterial tissue has been removed as possible with manual dissection. Regenerative cells can be obtained from the dissected cord lining by culturing the longitudinal pieces of cord lining on a fibronectin coated solid substrate (e.g., a plastic culture device such as a chambered slide or culture flask). The gelatinous surface of the cord lining can be placed in contact with the fibronectin coated solid substrate while the upper surface (i.e., the surface not in contact with the fibronectin coated solid substrate) can be covered with a solid substrate such as a coverslip. Low glucose (i.e., .1toreq.1 g/L glucose) growth medium can be added and the culture device incubated for a time sufficient for cells to migrate from the cord lining to the fibronectin coated solid substrate (e.g., 7 to 10 days). Unless otherwise indicated, cells are cultured at 37.degree. C. in a standard atmosphere that includes 5% CO.sub.2. Relative humidity is maintained at about 100%. After have adhered to the surface of the fibronectin coated solid substrate, the coverslip can be removed, and the adhered cells can be washed in a buffer such as phosphate-buffered saline (PBS). A growth medium that can be used for culturing Regenerative cellsis low glucose Dulbecco's Modified Essential Media (DMEM) containing vitamins (choline chloride, D-Calcium pantothenate, Folic Acid, Nicotinamide, Pyridoxal hydrochloride, Riboflavin, Thiamine hydrochloride, and i-Inositol), and non-essential amino acids (glycine, L-alanine, L-Asparagine, L-Aspartic acid, L-Glutamic Acid, L-Proline, and L-Serine). Low glucose DMEM can be supplemented with 10% to 20% serum (e.g., fetal bovine serum (FBS) or human serum), one or more antibiotics (e.g., gentamycin, penicillin, or streptomycin), and glutamine or a stabilized dipeptide of L-alanyl-L-glutamine (e.g., GlutaMax from Invitrogen). In one embodiment, a growth medium can include low glucose DMEM containing vitamins and non-essential amino acids, 15% FBS, 1 to 3% antibiotic (e.g., 2% or 2.times. gentamycin), and 0.7 to 1.5% (e.g., 1%) of glutamine or a stabilized dipeptide of L-alanyl-L-glutamine. Such a growth medium can be further supplemented with 1 to 100 ng/mL of a growth factor (e.g., basic fibroblast growth factor (bFGF), leukemia inhibitory factor (LIF), or epidermal growth factor (EGF).

In some embodiments, a growth medium further includes insulin, transferrin, selenium, and sodium pyruvate. A particularly useful growth medium can include low glucose DMEM containing vitamins and non-essential amino acids, 15% serum, 1 to 3% antibiotic (e.g., 2% or 2.times. gentamycin), 0.7 to 1.5% of glutamine or a stabilized dipeptide of L-alanyl-L-glutamine (e.g., 1% or 1.times. GlutaMax), 1 to 100 ng/mL of a growth factor (e.g., 10 ng/mL bFGF and 10 ng/mL LIF), 0.1 mg/mL to 100 mg/mL of insulin (10 mg/mL), 0.1 mg/mL to 100 mg/mL of transferrin (e.g., 0.55 mg/mL transferring), 0.1 .mu.g/mL to 100 .mu.g/mL selenium (e.g., 0.5 .mu.g/mL selenium), and 0.5 to 1.5% sodium pyruvate (e.g., 1% sodium pyruvate). In some embodiments, such a growth medium further includes 0.05 .mu.g/mL to 100 .mu.g/mL of putrescine (e.g., 10 .mu.g/mL putrescine) and 10 ng/mL of EGF. For embodiments in which an animal free medium is desired, human serum (e.g., 15% human serum) can be used in place of fetal bovine serum.

In some embodiments of the invention, it is necessary to subculture regenerative cells, TrypZean (Sigma Chemical Co.) can be used to release cells from the solid substrate. The resulting cell suspension can be pelleted and washed with PBS, then seeded into cell culture flasks at approximately 1000 cells/cm.sup.2 in a growth medium. Clonal lines of Regenerative cells can be established by plating the cells at a high dilution and using cloning rings (e.g., from Sigma) to isolate single colonies originating from a single cell. Cells are obtained from within the cloning ring using trypsin then re-plated in one well of a multi-well plate (e.g., a 6-well plate). After cells reach >60% confluency (e.g., >70% confluency), the cells can be transferred to a larger culture flask for further expansion. Regenerative cells can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

Regenerative cells can be immunophenotypically characterized using known techniques. For example, the cells can be fixed (e.g., in paraformaldehyde), permeabilized, and reactive sites blocked (e.g., with serum albumin), then incubated with an antibody having binding affinity for a cell surface antigen. The antibody can be detectably labeled (e.g., fluorescently or enzymatically) or can be detected using a secondary antibody that is detectably labeled. In some embodiments, the cell surface antigens on Regenerative cells can be characterized using flow cytometry and fluorescently labeled antibodies.

Regenerative cells also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining)

Regenerative cellscan be cryopreserved by suspending the cells (e.g., up to 5.times.10.sup.6 cells/mL) in a cryopreservative such as dimethylsulfoxide (DMSO, typically 10%). In some embodiments, a freezing medium such as CryoStor from Biolife solutions is used to cryopreserve the cells. After adding cryopreservative, the cells can be frozen (e.g., to −90.degree. C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90.degree. C., they can be placed in the liquid phase of the liquid nitrogen storage tank for long term storage. Cryopreservation can allow for long-term storage of these cells for therapeutic use.

These cells isolated according to the above methodology may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In particular embodiments, cells of the invention lack expression of CD90, CD105 and CD34 but possess CD56, and/or CD73 expression.

In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell. Exosomes produced by cells described herein, in some embodiments lack expression of miR

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In yet another aspect of the present disclosure, a method of treating a medical condition responsive to treatment with the isolated cells described herein can include introducing such cells into an individual having the medical condition. These cellular treatments can be utilized to treat any condition for which they are capable providing a benefit. Non-limiting examples of such medical conditions include COPD, diabetes, ischemia, osteoarthritis, orthopedic damage, liver damage, chronic refractory angina, erectile dysfunction, herniated disks, congestive heart failure, asthma, emphysema, wounds, acute radiation syndrome, autoimmune disorders, ischemic organ beds, graft vs. host disease, and the like, including combinations thereof. Additionally, in another aspect, a method of treating a medical condition responsive to treatment with the differentiated cells described herein can include introducing at least one cell type of the differentiated cells into an individual having the medical condition.

### EXAMPLES

### Example 1: Protection from Radiation Induced Death

All umbilical cords were obtained from clinically normal pregnancies after signing of informed consent. Umbilical cord tissue was washed with Hank's balanced salt solution (HBSS) (Hyclone, Rochester, NY). The cords were dissected; care was taken to remove the artery, veins, and Wharton's jelly. For histological analysis, a 10-mm piece of umbilical cord was placed into 4% paraformaldehyde. The remaining subepithelial layer was then placed into Dulbecco's phosphate-buffered saline (DPBS) containing 10% XcyteMLPL™ supplement (JadiCell, Salt Lake City, UT). The subepithelial layer was cut into 10×10-mm sections and placed into six-well dishes. A 22×22-mm sterile coverslip (Fisher Scientific, Pittsburgh, PA) was placed over the subepithelial layer. Explants were cultured in Dulbecco's modified Eagle's medium (DMEM) low glucose, 10% XcyteMLPL™ supplement, 1× Glutamax, and 1× minimum essential medium with nonessential amino acids (MEM-NEAA; Life Technologies) and cultured in 5% CO2 incubators at 37° C. After 2 days, the cells are 70% confluent and non-enzymatic xeno-free seperation using trypLE (Life Technologies). Cells were passaged onto T225 flasks at a density of 1,000 cells/cm2

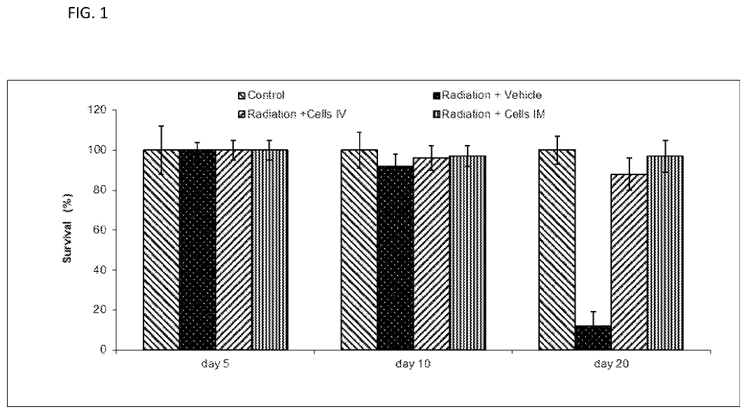

Cells were subsequently used to treat animals exposed to radiation 1 day after exposure. After the required acclimatization period of >3 days at the animal colony, the mice were exposed to total body irradiation (TBI) in pathogen free conditions with photon beam of clinical 6 MeV LINAC. The mice were placed in a plastic restricting Plexiglas jig, and 8 Gy was delivered through 5-mm plastic build-up for homogenous dose distribution. Cells from 5-8 passages were harvested for injection from confluent flasks using trypLE-Versene EDTA solution. The cells were re-suspended in medium, counted, and centrifuged by 1400 csf for 5 min at 4° C. Then the cells were re-suspended in plasmaLyte A (designated also as “Vehicle”) to reach a final concentration of 2×106 cells/100 μl. Two 50-μl injections of 106 cells were delivered in each treatment intramuscularly (IM) or intravenously (IV), 1 day following 8-Gy TBI. In the IM treatment, the cells were injected to the large muscles of both hind legs, and in IV treatment they were injected via tail vein at the same time points. Results are shown in FIG. 1.

### Example 2: Subepithelial Lining of Umbilical Cord Cells Stimulates Hematopoietic Cytokine VEGF

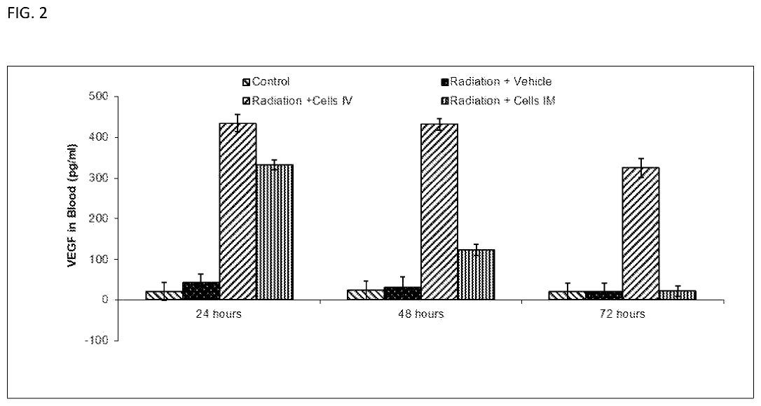

Animals from Example 1 were assessed for expression of VEGF on the indicated timepoints. VEGF is measured by ELISA of blood. Results are shown in FIG. 2.

### Example 3: Subepithelial Lining of Umbilical Cord Cells Stimulates Hematopoietic Cytokine HGF-1

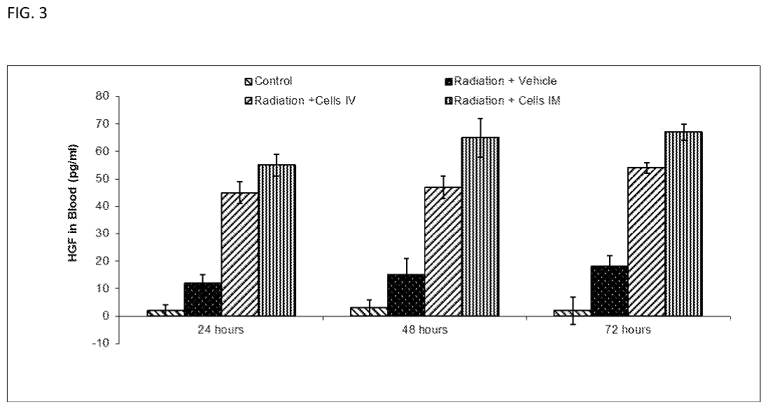

Animals from Example 1 were assessed for expression of HGF-1 on the indicated timepoints. HGF-1 is measured by ELISA of blood. Results are shown in FIG. 3.

### Example 4: Subepithelial Lining of Umbilical Cord Cells Stimulates Hematopoietic Cytokine Angiopoietin

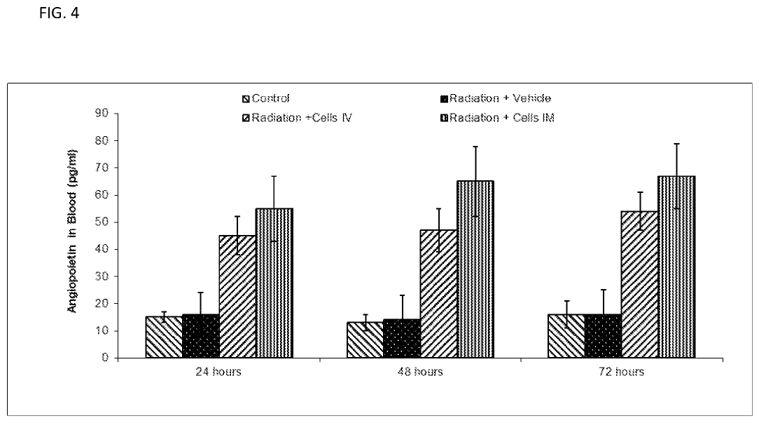

Animals from Example 1 were assessed for expression of angiopoietin on the indicated timepoints. Angiopoietin is measured by ELISA of blood. Results are shown in FIG. 4.

### Example 5: Subepithelial Lining of Umbilical Cord Cells Stimulates Hematopoietic Cytokine M-CSF

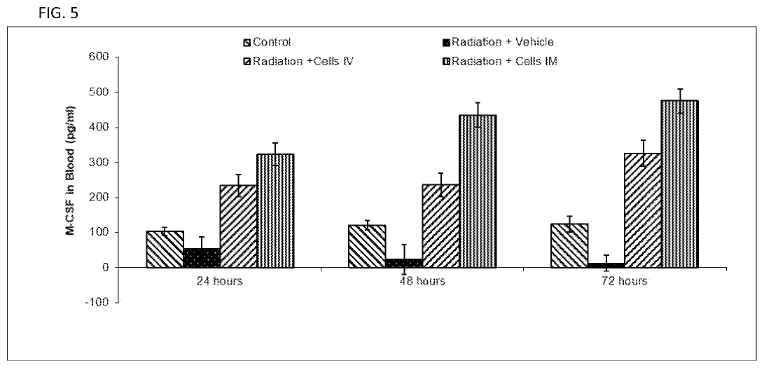

Animals from Example 1 were assessed for expression of M-CSF on the indicated timepoints. Angiopoietin is measured by ELISA of blood. Results are shown in FIG. 5

### Example 6: Subepithelial Lining of Umbilical Cord Cells Stimulates Hematopoietic Cytokine G-CSF

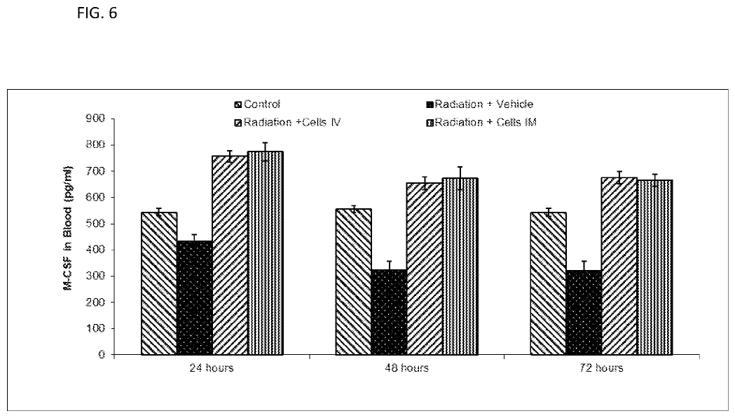

Animals from Example 1 were assessed for expression of G-CSF on the indicated timepoints. Angiopoietin is measured by ELISA of blood. Results are shown in FIG. 6.

### Example 7: Subepithelial Lining of Umbilical Cord Cells Stimulates Hematopoietic Cytokine GM-CSF

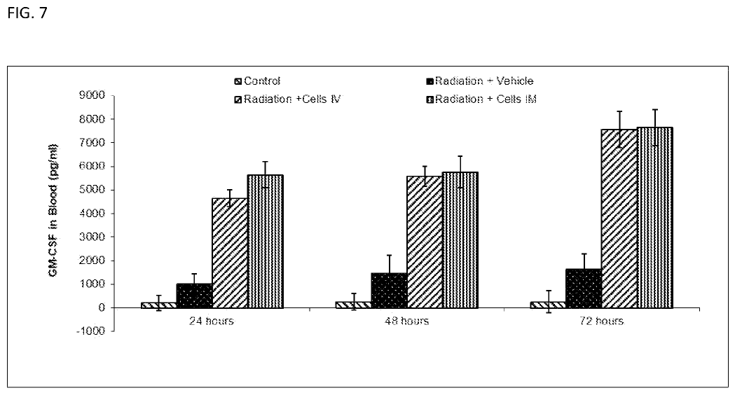

Animals from Example 1 were assessed for expression of GM-CSF on the indicated timepoints. Angiopoietin is measured by ELISA of blood. Results are shown in FIG. 7.

### REFERENCES

1. Kumar, A., et al., Role of melatonin mediated G-CSF induction in hematopoietic system of gamma-irradiated mice. Life Sci, 2022. 289: p. 120190.
2. Kim, L. N., et al., Noninvasive Systemic Modalities for Prevention of Head and Neck Radiation-Associated Soft Tissue Injury: A Narrative Review. J Reconstr Microsurg, 2022.
3. Alidadi, S., et al., The efficacy of melatonin against radiotoxicity of iodine-131 and its response to treatment in hyperthyroid patients: a randomized controlled trial. Nucl Med Rev Cent East Eur, 2022. 25(1): p. 31-36.
4. Garcia-Segura, L. C., et al., Compounded Melatonin Cream for the Prevention and Treatment of Radiation Dermatitis: A Case Report. Int J Pharm Compd, 2022. 26(1): p. 6-8.
5. Hu, W., et al., Melatonin attenuates radiation-induced cortical bone-derived stem cells injury and enhances bone repair in postradiation femoral defect model. Mil Med Res, 2021. 8(1): p. 61.
6. Samei, E., et al., Radioprotective Effects of Combined Melatonin and Famotidine Treatment on Radiation Induced Apoptosis in Peripheral Blood Leukocytes of Breast Cancer Patients and Normal Individuals. Cell J, 2021. 23(5): p. 562-567.
7. Minella, C., et al., Protective role of melatonin in breast cancer: what we can learn from women with blindness. Cancer Causes Control, 2022. 33(1): p. 1-13.
8. Wang, Z., Y. Liu, and A. E. Musa, Regulation of cell death mechanisms by melatonin: implications to cancer therapy. Anticancer Agents Med Chem, 2021.
9. Wang, Q., et al., The Effects of Melatonin Administration on Intestinal Injury Caused by Abdominal Irradiation from Mice. Int J Mol Sci, 2021. 22(18).
10. Jafarpour, S. M., et al., The Radioprotective Effects of Melatonin and Nanoselenium on DNA Double-Strand Breaks in Peripheral Lymphocytes Caused by I-131. Indian J Nucl Med, 2021. 36(2): p. 134-139.
11. Panciroli, C., et al., Prospective pilot study to explore the melatonin level in brain tumor patients undergoing radiotherapy. Sleep Breath, 2022. 26(1): p. 469-475.
12. Lozano, A., et al., Randomized placebo-controlled phase II trial of high-dose melatonin mucoadhesive oral gel for the prevention and treatment of oral mucositis in patients with head and neck cancer undergoing radiation therapy concurrent with systemic treatment. Clin Transl Oncol, 2021. 23(9): p. 1801-1810.
13. Nuszkiewicz, J., A. Wozniak, and K. Szewczyk-Golec, Ionizing Radiation as a Source of Oxidative Stress-The Protective Role of Melatonin and Vitamin D. Int J Mol Sci, 2020. 21(16).
14. Rakici, S. Y., et al., Radioprotective effect of endogenous melatonin secretion associated with the circadian rhythm in irradiated rats. Int J Radiat Biol, 2019. 95(9): p. 1236-1241.
15. Rezaeejam, H., et al., Radioprotective effect of melatonin on expression of Cdknla and Rad50 genes in rat peripheral blood. J Cancer Res Ther, 2018. 14(Supplement): p. S1070-S1075.
16. Shabeeb, D., et al., Biochemical and Histopathological Evaluation of the Radioprotective Effects of Melatonin Against Gamma Ray-Induced Skin Damage. Curr Radiopharm, 2019. 12(1): p. 72-81.
17. Kouhi Habibi, N., et al., The protective effects of melatonin on blood cell counts of rectal cancer patients following radio-chemotherapy: a randomized controlled trial. Clin Transl Oncol, 2019. 21(6): p. 745-752.
18. Farhood, B., et al., Melatonin as an adjuvant in radiotherapy for radioprotection and radiosensitization. Clin Transl Oncol, 2019. 21(3): p. 268-279.
19. Amini, P., et al., Mechanisms for Radioprotection by Melatonin; Can it be Used as a Radiation Countermeasure? Curr Mol Pharmacol, 2019. 12(1): p. 2-11.
20. Mihandoost, E., et al., Consequences of lethal-whole-body gamma radiation and possible ameliorative role of melatonin. ScientificWorldJournal, 2014. 2014: p. 621570.
21. Wang, Q., et al., Quantitative proteomic analysis of the effects of melatonin treatment for mice suffered from small intestinal damage induced by gamma-ray radiation. Int J Radiat Biol, 2021. 97(9): p. 1206-1216.
22. Uthaiwat, P., et al., Oral Administration of Melatonin or Succinyl Melatonin Niosome Gel Benefits 5-FU-Induced Small Intestinal Mucositis Treatment in Mice. AAPS PharmSciTech, 2021. 22(5): p. 200.
23. Sezen, O., et al., The role of melatonin in preventing radiation-induced intestinal injury. J BUON, 2021. 26(2): p. 626-633.
24. Sadeghi, H., et al., Mitigation of Radiation-Induced Gastrointestinal System Injury by Melatonin: A Histopathological Study. Curr Drug Res Rev, 2020. 12(1): p. 72-79.
25. Najafi, M., et al., The Radioprotective Effect of Combination of Melatonin and Metformin on Rat Duodenum Damage Induced by Ionizing Radiation: A Histological Study. Adv Biomed Res, 2019. 8: p. 51.
26. Najafi, M., et al., Protection from Radiation-induced Damage in Rat's Ileum and Colon by Combined Regimens of Melatonin and Metformin: A Histopathological Study. Antiinflamm Antiallergy Agents Med Chem, 2020. 19(2): p. 180-189.
27. Musa, A. E., D. Shabeeb, and H. S. Q. Alhilfi, Protective Effect of Melatonin Against Radiotherapy-Induced Small Intestinal Oxidative Stress: Biochemical Evaluation. Medicina (Kaunas), 2019. 55(6).
28. Fernandez-Gil, B., et al., Melatonin protects rats from radiotherapy-induced small intestine toxicity. PLoS One, 2017. 12(4): p. e0174474.
29. Sheikholeslami, S., et al., The role of melatonin on radiation-induced pneumonitis and lung fibrosis: A systematic review. Life Sci, 2021. 281: p. 119721.
30. Wang, S., et al., Protective effect of melatonin entrapped PLGA nanoparticles on radiation-induced lung injury through the miR-21/TGF-beta1/Smad3 pathway. Int J Pharm, 2021. 602: p. 120584.
31. Farhood, B., et al., Mitigation of Radiation-Induced Lung Pneumonitis and Fibrosis Using Metformin and Melatonin: A Histopathological Study. Medicina (Kaunas), 2019. 55(8).
32. Tahamtan, R., et al., Radioprotective effect of melatonin on radiation-induced lung injury and lipid peroxidation in rats. Cell J, 2015. 17(1): p. 111-20.
33. Chen, T., et al., Melatonin reduces radiation damage in inner ear. J Radiat Res, 2021. 62(2): p. 217-225.
34. Karaer, I., et al., Melatonin protects inner ear against radiation damage in rats. Laryngoscope, 2015. 125(10): p. E345-9.
35. Uthaiwat, P., et al., Topical Melatonin Niosome Gel for the Treatment of 5-FU-Induced Oral Mucositis in Mice. Curr Drug Deliv, 2021. 18(2): p. 199-211.
36. Elsabagh, H. H., et al., Efficacy of Melatonin in prevention of radiation-induced oral mucositis: A randomized clinical trial. Oral Dis, 2020. 26(3): p. 566-572.
37. Ortiz, F., et al., Melatonin blunts the mitochondrial/NLRP3 connection and protects against radiation-induced oral mucositis. J Pineal Res, 2015. 58(1): p. 34-49.
38. Shabeeb, D., et al., Evaluation of the Radioprotective Effects of Melatonin Against Ionizing Radiation-Induced Muscle Tissue Injury. Curr Radiopharm, 2019. 12(3): p. 247-255.
39. Amini, P., et al., Mitigation of radiation-induced hematopoietic system injury by melatonin. Environ Toxicol, 2020. 35(8): p. 815-821.
40. Mao, A., et al., Exogenous melatonin modulates carbon ion radiation-induced immune dysfunction in mice. Toxicology, 2019. 417: p. 35-41.
41. Khan, S., et al., Melatonin attenuates (60) Co gamma-ray-induced hematopoietic, immunological and gastrointestinal injuries in C57BL/6 male mice. Environ Toxicol, 2017. 32(2): p. 501-518.
42. Tajabadi, E., et al., Radioprotective effect of a combination of melatonin and metformin on mice spermatogenesis: A histological study. Int J Reprod Biomed, 2020. 18(12): p. 1073-1080.
43. Pandey, N. and S. Giri, Melatonin attenuates radiofrequency radiation (900 MHz)-induced oxidative stress, DNA damage and cell cycle arrest in germ cells of male Swiss albino mice. Toxicol Ind Health, 2018. 34(5): p. 315-327.
44. Gholami, M., et al., Protective effects of melatonin and ghrelin on spermatogenesis: A narrative review of the literature. Int J Reprod Biomed, 2017. 15(5): p. 265-272.
45. Khan, S., et al., Radioprotective potential of melatonin against (6)(0)Co gamma-ray-induced testicular injury in male C57BL/6 mice. J Biomed Sci, 2015. 22: p. 61.
46. Aricigil, M., et al., Melatonin prevents possible radiotherapy-induced thyroid injury. Int J Radiat Biol, 2017. 93(12): p. 1350-1356.
47. Canyilmaz, E., et al., Comparison of the effects of melatonin and genistein on radiation-induced nephrotoxicity: Results of an experimental study. Biomed Rep, 2016. 4(1): p. 45-50.
48. Kucuktulu, E., Protective effect of melatonin against radiation induced nephrotoxicity in rats. Asian Pac J Cancer Prey, 2012. 13(8): p. 4101-5.
49. Aras, S., et al., Radioprotective effect of melatonin against radiotherapy-induced cerebral cortex and cerebellum damage in rat. Int J Radiat Biol, 2021. 97(3): p. 348-355.
50. Pipova Kokosova, N., et al., Melatonin mitigates hippocampal and cognitive impairments caused by prenatal irradiation. Eur J Neurosci, 2020. 52(6): p. 3575-3594.
51. Shabeeb, D., et al., Histopathological and Functional Evaluation of Radiation-Induced Sciatic Nerve Damage: Melatonin as Radioprotector. Medicina (Kaunas), 2019. 55(8).
52. Naseri, S., et al., Radio-Protective Effects of Melatonin on Subventricular Zone in Irradiated Rat. Decrease in Apoptosis and Upregulation of Nestin. J Mol Neurosci, 2017. 63(2): p. 198-205.
53. Haridas, S., M. Kumar, and K. Manda, Chronic melatonin administration mitigates behavioral dysfunction induced by gamma-irradiation. Horm Behav, 2012. 62(5): p. 621-7.
54. Hanedan Uslu, G., et al., Protective effects of genistein and melatonin on mouse liver injury induced by whole-body ionising radiation. Mol Clin Oncol, 2019. 10(2): p. 261-266.
55. Najafi, M., et al., Melatonin as an anti-inflammatory agent in radiotherapy. Inflammopharmacology, 2017. 25(4): p. 403-413.
56. Cakir, Z. U., et al., Melatonin can Ameliorate Radiation-Induced Oxidative Stress and Inflammation-Related Deterioration of Bone Quality in Rat Femur. Inflammation, 2016. 39(3): p. 1134-40.
57. Najafi, M., et al., The melatonin immunomodulatory actions in radiotherapy. Biophys Rev, 2017. 9(2): p. 139-148.
58. Fatfat, Z., M. Fatfat, and H. Gali-Muhtasib, Therapeutic potential of thymoquinone in combination therapy against cancer and cancer stem cells. World J Clin Oncol, 2021. 12(7): p. 522-543.
59. Nemec-Bakk, A. S., et al., Mitigation of late cardiovascular effects of oxygen ion radiation by gamma-tocotrienol in a mouse model. Life Sci Space Res (Amst), 2021. 31: p. 43-50.
60. Kumar, V. P., et al., Gamma Tocotrienol Protects Mice From Targeted Thoracic Radiation Injury. Front Pharmacol, 2020. 11: p. 587970.
61. Lee, S. G., et al., gamma-Tocotrienol-Loaded Liposomes for Radioprotection from Hematopoietic Side Effects Caused by Radiotherapeutic Drugs. J Nucl Med, 2021. 62(4): p. 584-590.
62. Pathak, R., et al., Enhanced Survival in Mice Exposed to Ionizing Radiation by Combination of Gamma-Tocotrienol and Simvastatin. Mil Med, 2019. 184(Suppl 1): p. 644-651.

63. Cheema, A. K., et al., Proteomic Changes in Mouse Spleen after Radiation-Induced Injury and its Modulation by Gamma-Tocotrienol. Radiat Res, 2018. 190(5): p. 449-463.
64. Pannkuk, E. L., et al., A Metabolomic Serum Signature from Nonhuman Primates Treated with a Radiation Countermeasure, Gamma-tocotrienol, and Exposed to Ionizing Radiation. Health Phys, 2018. 115(1): p. 3-11.
65. Banerjee, S., et al., Cebpd Is Essential for Gamma-Tocotrienol Mediated Protection against Radiation-Induced Hematopoietic and Intestinal Injury. Antioxidants (Basel), 2018. 7(4).
66. Ledet, G. A., et al., Development of Orally Administered gamma-Tocotrienol (GT3) Nanoemulsion for Radioprotection. Int J Mol Sci, 2016. 18(1).
67. Singh, V. K., et al., The Potentiation of the Radioprotective Efficacy of Two Medical Countermeasures, Gamma-Tocotrienol and Amifostine, by a Combination Prophylactic Modality. Radiat Prot Dosimetry, 2016. 172(1-3): p. 302-310.
68. Singh, V. K., et al., Progenitor Cell Mobilization by Gamma-tocotrienol: A Promising Radiation Countermeasure. Health Phys, 2016. 111(2): p. 85-92.
69. Pathak, R., et al., The Vitamin E Analog Gamma-Tocotrienol (GT3) Suppresses Radiation-Induced Cytogenetic Damage. Pharm Res, 2016. 33(9): p. 2117-25.
70. Singh, V. K. and M. Hauer-Jensen, gamma-Tocotrienol as a Promising Countermeasure for Acute Radiation Syndrome: Current Status. Int J Mol Sci, 2016. 17(5).
71. Ghosh, S. P., et al., Gamma-Tocotrienol Modulates Radiation-Induced MicroRNA Expression in Mouse Spleen. Radiat Res, 2016. 185(5): p. 485-95.
72. Singh, V. K., et al., Radioprotective Efficacy of Gamma-Tocotrienol in Nonhuman Primates. Radiat Res, 2016. 185(3): p. 285-98.
73. Singh, V. K., et al., Progenitors mobilized by gamma-tocotrienol as an effective radiation countermeasure. PLoS One, 2014. 9(11): p. el14078.
74. Singh, V. K., et al., Radioprotective efficacy of delta-tocotrienol, a vitamin E isoform, is mediated through granulocyte colony-stimulating factor. Life Sci, 2014. 98(2): p. 113-22.
75. Suman, S., et al., Gamma tocotrienol, a potent radioprotector, preferentially upregulates expression of anti-apoptotic genes to promote intestinal cell survival. Food Chem Toxicol, 2013. 60: p. 488-96.
76. Kulkarni, S., et al., Granulocyte colony-stimulating factor antibody abrogates radioprotective efficacy of gamma-tocotrienol, a promising radiation countermeasure. Cytokine, 2013. 62(2): p. 278-85.
77. Ray, S., et al., Mobilization of progenitor cells into peripheral blood by gamma-tocotrienol: a promising radiation countermeasure. Int Immunopharmacol, 2013. 15(3): p. 557-64.
78. Berbee, M., et al., Reduction of radiation-induced vascular nitrosative stress by the vitamin E analog gamma-tocotrienol: evidence of a role for tetrahydrobiopterin. Int J Radiat Oncol Biol Phys, 2011. 79(3): p. 884-91.
79. Kulkarni, S., et al., Gamma-tocotrienol protects hematopoietic stem and progenitor cells in mice after total-body irradiation. Radiat Res, 2010. 173(6): p. 738-47.
80. Berbee, M., et al., gamma-Tocotrienol ameliorates intestinal radiation injury and reduces vascular oxidative stress after total-body irradiation by an HMG-CoA reductase-dependent mechanism. Radiat Res, 2009. 171(5): p. 596-605.
81. Andreyev, H. J. N., et al., Randomised single centre double-blind placebo controlled phase II trial of Tocovid SupraBio in combination with pentoxifylline in patients suffering long-term gastrointestinal adverse effects of radiotherapy for pelvic cancer: The PPALM study. Radiother Oncol, 2022. 168: p. 130-137.
82. Berbee, M., et al., Pentoxifylline enhances the radioprotective properties of gamma-tocotrienol: differential effects on the hematopoietic, gastrointestinal and vascular systems. Radiat Res, 2011. 175(3): p. 297-306.
83. Boutros, S. W., et al., Amifostine (WR-2721) Mitigates Cognitive Injury Induced by Heavy Ion Radiation in Male Mice and Alters Behavior and Brain Connectivity. Front Physiol, 2021. 12: p. 770502.
84. Cheema, A. K., et al., Alterations in Tissue Metabolite Profiles with Amifostine-Prophylaxed Mice Exposed to Gamma Radiation. Metabolites, 2020. 10(5).
85. Ozdemir, C. S., et al., The effect of Amifostine (Ethyol) on intestinal anastomosis in rats with radiation enteritis. Eur Rev Med Pharmacol Sci, 2013. 17(10): p. 1351-9.
86. Benkovic, V., et al., Potential radioprotective properties of arbutin against ionising radiation on human leukocytes in vitro. Mutat Res Genet Toxicol Environ Mutagen, 2021. 872: p. 503413.
87. Zhou, W., et al., The Protective Effect of Rosavin from Rhodiola rosea on Radiation-Induced Intestinal Injury. Chem Biodivers, 2020. 17(12): p. e2000652.
88. S, N., et al., Radioprotective Effect of Arbutin in Megavoltage Therapeutic X-irradiated Mice using Liver Enzymes Assessment. J Biomed Phys Eng, 2019. 9(5): p. 533-540.
89. Nadi, S., et al., Effects of Arbutin on Radiation-Induced Micronuclei in Mice Bone Marrow Cells and Its Definite Dose Reduction Factor. Iran J Med Sci, 2016. 41(3): p. 180-5.
90. Godevac, D., et al., Phenolic Compounds from Atriplex littoralis and Their Radiation-Mitigating Activity. J Nat Prod, 2015. 78(9): p. 2198-204.
91. Adenan, M. N. H., et al., Radioprotective Effects of Kelulut Honey in Zebrafish Model. Molecules, 2021. 26(6).

1. A method of treating radiation exposure comprising administering a therapeutic dose of regenerative cells derived from the subepithelial area of the umbilical cord/perinatal tissue to a patient having been exposed to radiation.

2. The method of claim 1, wherein said radiation exposure is associated with damage to the hematopoietic system.

3. The method of claim 2, wherein said damage to the hematopoietic system is reduction suppression of ability of bone marrow stromal cells to produce growth factors.

4. The method of claim 3, wherein said growth factors whose production is reduced by radiation damage are selected from the group consisting of: BLC, Eotaxin-1, Eotaxin-2, G-CSF, GM-CSF, 1-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HBEGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFNgammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163 , Clusterin, CRTAM, CXCL14, Cystatin C, Decorin , Dkk-3, DLL1, Fetuin A, aFGF, FOLR1, Furin, GASP-1, GASP-2, GCSF R, HAI-2, IL-17B R, IL-27, LAG-3 , LDL R, Pepsinogen I, RBP4, SOST, Syndecan-1, TACI, TFPI, TSP-1, TRAIL R2 , TRANCE, Troponin I, uPA, VE-Cadherin, WISP-1, and RANK. ANG, EGF, ENA-78, FGF2, Follistatin, G-CSF, GRO, HGF, IL-6, IL-8, Leptin, MCP-1, MCP-3, PDGFB, PLGF, Rantes, TGFBI, Thrombopoietin, TIMPI, TIMP2, uPAR, VEGF, VEGFD, angiopoietin-1, and angiopoietin-2.

5. The method of claim 1, wherein said radiation exposure is associated with damage to the gastrointestinal system.

6. The method of claim 1, wherein said radiation exposure is associated with damage to the respiratory system.

7. The method of claim 1, wherein derivatives of said regenerative cell are utilized for radioprotection.

8. The method of claim 7, wherein said derivates are selected from the group consisting of: a) exosomes; b) apoptotic bodies; c) extracellular vesicles; and d) conditioned media.

9. The method of claim 1, wherein said cells are treated with an activator of an immune receptor.

10. The method of 9, wherein said immune receptor is TLR. 1.

11. The method of claim 1, wherein said cells express GM-CSF receptor.

12. The method of claim 1, wherein said cells express surface vimentin and a marker selected from the group consisting of: a) CD29; b) CD36; c) CD37; d) CD73; e) CD90; f) CD166; g) SSEA4; h) CD9; i) CD44; k) CD146; 1) CD105; and m) HLA-G

13. The method of claim 12, wherein said cells possess ability to generate soluble TNF-alpha receptor at a concentration of 10 pg-1 ng per 1,000,000 cells under basal growth conditions in DMEM media with 10% fetal calf serum.

14. The method of claim 1, wherein said cell population expresses interleukin-6 receptor and CD73.

15. The method of claim 1, wherein said patient exposed to radiation is also treated in a manner to alter the gut microbiome in order to obtain enhanced protective and/or regenerative effects from radiation exposure.

16. The composition of claim 1, wherein said cells are obtained from placenta perivascular tissue as a substitute for Wharton's Jelly.

17. The composition of claim 1, wherein said regenerative adjuvant is an anti-inflammatory cytokine.

18. The composition of claim 17, wherein said anti-inflammatory cytokine is selected from the group consisting of IL-4, IL-10, IL-13, IL-20, IL-22 and IL-35.

19. The composition of claim 18, wherein said anti-inflammatory cytokine is VEGF.

20. The composition of claim 1, wherein said composition is capable of inhibiting T cell mediated immune responses.
